(19) United States
(12) Patent Application Publication
(10) Pub. No.: US 2024/0188196 A1
(43) Pub. Date: Jun. 6, 2024

(54) INDUCTION HEATER MODULE AND ADJUSTMENT DEVICE FOR INDUCTION HEATING

(71) Applicants: George R. Breiwa, III, Dane, WI (US); Christopher L. Bailey, Louisville, KY (US); Pranav Sawant, Madison, WI (US)

(72) Inventors: George R. Breiwa, III, Dane, WI (US); Christopher L. Bailey, Louisville, KY (US); Pranav Sawant, Madison, WI (US)

(21) Appl. No.: 18/282,459

(22) PCT Filed: Mar. 18, 2022
(86) PCT No.: PCT/US2022/020927

§ 371 Date: Sep. 15, 2023

Related U.S. Application Data
(60) Provisional application No. 63/166,601, filed on Mar. 26, 2021.
(60) Provisional application No. 63/230,887, filed on Aug. 9, 2021.
(60) Provisional application No. 63/250,679, filed on Sep. 30, 2021.
(60) Provisional application No. 63/292,311, filed on Dec. 21, 2021.

(51) Int. Cl. H05B 6/10

(52) U.S. Cl. CPC H05B 6/108

(57) ABSTRACT

An induction heater module is disclosed comprising a (510) threaded connector couplable to a power source, an induction circuit electrically connected to the (510) threaded connector, and an induction coil electrically connected to the induction circuit. An incremental ingression insert adjustment device is also disclosed comprising, a receptacle having an inner recess for receipt of an administration device, and an exterior surface comprising a thread having a pitch aligned with the pitch of an induction coil of an induction heater, wherein the receptacle is rotatably or insatiably engaged with the induction coil. A base for an induction heater module and a housing for an induction heater module are also disclosed.

### CROSS-REFERENCE TO RELATED APPLICATIONS

This application claims priority to U.S. Provisional Patent Application, Ser. No. 63/166,601, filed Mar. 26, 2021, entitled “Induction Heater with 510 Connector,” to U.S. Provisional Patent Application, Ser. No. 63/230,887, filed Aug. 9, 2021, entitled “Induction Heater Depth Control Device,” to U.S. Provisional Patent Application, Ser. No. 63/250,679, filed Sep. 30, 2021, entitled “Induction Heater Module Housing,” and to U.S. Provisional Patent Application, Ser. No. 63/292,311, filed Dec. 21, 2021, entitled “Incremental Ingression Insert Adjustment Device for an Induction Heater,” the entire contents of each of which are hereby incorporated by reference herein in their entirety.

### BACKGROUND

The disclosure herein relates to the field of induction heaters. The disclosure more specifically relates to the field of induction heater modules, accessories and attachments for induction heaters.

Induction heaters are known. Induction heaters are designed to heat in response to the presence of a metal material within an induction coil.

An induction heater is a heating device for an administration device that uses induction to generate heat within the administration device. Such products are often used with thermal extraction, smoking, and vaping devices. Generally, the induction heater is comprised of a power source connected to the appropriate circuitry for creating an oscillating electromagnetic field. This field is concentrated with an induction coil situated in a position to allow for easy heating of the administration device. Induction heating in the induction heater is caused by a rapidly oscillating electric field in one or more coils of wire that generate(s) current in certain metals placed within the coil. Because the metal of the administration device has electrical resistance, the current induced will generate heat. The heat is generated from within the metal being heated.

Portable induction heaters, while portable, are generally larger than a smartphone and can be cumbersome. These devices typically consist of a power source, circuitry, an induction coil, and other features, such as switches, lights, displays, and controls. A need exists for a small induction heating device that is easy to manufacture, has fewer components which could fail, and is user customizable.

In addition, it has been learned that insertion depth is an important feature for accurate heating of the administration device with an induction heater. Currently, a user inserts the administration device into an aperture of standard depth surrounded by an induction coil. When heating an administration device, the location of the focal point of the applied heat may have an impact on the quality of the heating. If heat is applied too close to the tip or base of the administration device, the material to be extracted may be over or under heated. Consequently, finding the ideal focal point impacts the effectiveness of the extraction and may significantly improve the user experience. Therefore, a need also exists for a means to control the insertion depth or ingress of the administration device into the induction coil.

### SUMMARY

Accordingly, an induction heater module is disclosed. The induction heater module comprises an induction heating circuit in electrical communication with a 510 electrical coupling and in electrical communication with an induction coil which receives an incremental ingression insert adjustment device.

An induction heater module is also disclosed comprising a 510 threaded connector couplable to a power source, an induction circuit electrically connected to the 510 threaded connector, and an induction coil electrically connected to the induction circuit.

A base for an induction heater module housing is also disclosed, the base comprises a conductive material, a 510 threaded connection which allows for connection to a power supply, and a base geometry enabling simplistic, manufacturing of a couplable housing.

A base of an induction heater module is also disclosed which comprises a 510 threaded connector couplable to a power source and electrically couplable to an induction heating circuit and induction coil. The base comprises a geometry which enables press fit attachment of a housing body.

An induction heater module housing is disclosed. The induction heater module housing comprises a base couplable to a housing body on a first end, and a cover or lid couplable to the housing body on a second end, wherein the housing receives an induction heating circuit and coil and a receptacle for receipt of a thermal extraction device or administration device.

An induction heater module housing is also disclosed. The housing includes a base with an electrical coupling. The base is couplable to a housing body on a first end of the housing body. A cover is couplable to the housing body on a second end of the housing body.

An induction heater depth control device is further disclosed. More specifically, an incremental ingression insert adjustment device for an induction heater is disclosed. The device comprises a receptacle having an inner recess for receipt of an administration device to be heated, and an exterior surface comprising a thread having a pitch aligned with the pitch of an induction coil of an induction heater.

An incremental ingression insert adjustment device is also disclosed comprising, a receptacle having an inner recess for receipt of an administration device, and an exterior surface comprising a thread having a pitch aligned with the pitch of an induction coil of an induction heater, wherein the receptacle is rotatably or insertably engaged with the induction coil.

These and other features and advantages of devices, systems, and methods are described in, or are apparent from, the following detailed descriptions and drawings of various examples of embodiments.

### BRIEF DESCRIPTION OF DRAWINGS

Various examples of embodiments of the systems, devices, and methods will be described in detail, with reference to the following figures, wherein:

FIG. 1 is a perspective view of an induction heater module on a power supply according to one or more examples of embodiments.

FIG. 2 is a front elevation view of the induction heater module on a power supply shown in FIG. 1.

FIG. 3 is an exploded perspective view of an induction heater module according to one or more examples of embodiments.

FIG. 4 is an additional exploded perspective view of an induction heater module according to one or more examples of embodiments.

FIG. 5 is a electrical connection block diagram of the induction heating module according to one or more examples of embodiments.

FIG. 6 is a perspective view of an administration device or thermal extraction device for use with the induction heater module described herein.

FIG. 7 is a perspective view of an induction heater module housing according to one or more examples of embodiments.

FIG. 8 is a second perspective view of an induction heater module housing according to one or more examples of embodiments.

FIG. 9 is an exploded perspective view of the induction heater module housing shown in FIGS. 7-8.

FIG. 10 is a perspective view of a base showing a 510 connector, the base being for use with the induction heater module in one or more examples of embodiments.

FIG. 11 is a bottom elevation view of the base shown in FIG. 10, also showing housing body attached thereto.

FIG. 12 is an additional perspective view of the base for use with the induction heater module in one or more examples of embodiments, showing the electrical coupling surface for coupling to an induction circuit.

FIG. 13 is a perspective view of a housing body for use with the induction heater module in one or more examples of embodiments.

FIG. 14 is an additional perspective view of the housing body shown in FIG. 13.

FIG. 15 is a perspective view of a cover for use with the induction heater module in one or more examples of embodiments.

FIG. 16 is a plan view of the cover shown in FIG. 15.

FIG. 17 is a perspective view of a power source, namely a base station, for use with the induction heater module in one or more examples of embodiments.

FIG. 18 is an exploded perspective view of a power source, namely a base station, and induction heater module according to one or more examples of embodiments.

FIG. 19 is a perspective view of a power source, namely a base station, and induction heater module of FIG. 18, joined together, according to one or more examples of embodiments.

FIG. 20 is a perspective view of an incremental ingression insert adjustment device according to one or more examples of embodiments.

FIG. 21 is a perspective view of one or more alternative examples of embodiments of an incremental ingression insert adjustment device and an induction coil.

FIG. 22 is a side elevation view of an incremental ingression insert adjustment device and an induction coil.

FIG. 23 is a side elevation view of an incremental ingression insert adjustment device and an induction coil, showing the incremental ingression insert adjustment device inserted into the induction coil.

FIG. 24 is a perspective view showing the incremental ingression insert adjustment device inserted into the induction coil of FIG. 23 electrically coupled to an induction circuit.

It should be understood that the drawings are not necessarily to scale. In certain instances, details that are not necessary to the understanding of the invention or render other details difficult to perceive may have been omitted. For ease of understanding and simplicity, common numbering of elements within the numerous illustrations is utilized when the element is the same in different Figures. It should be understood, of course, that the invention is not necessarily limited to the particular embodiments illustrated herein.

### DETAILED DESCRIPTION

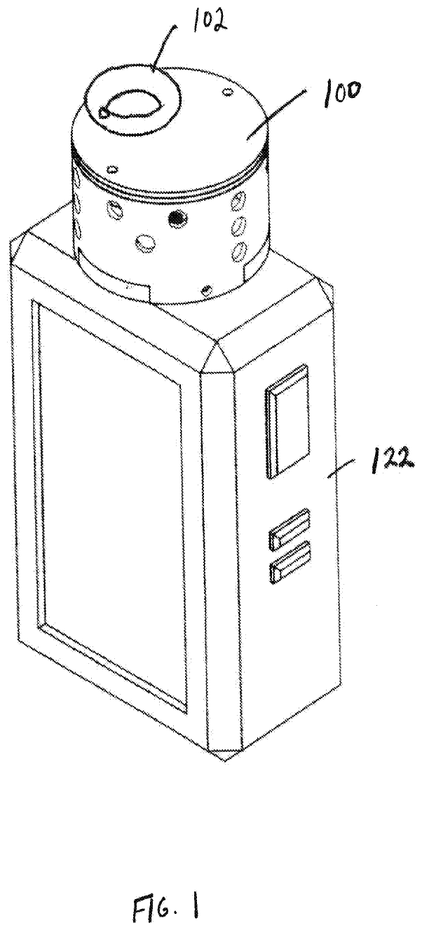

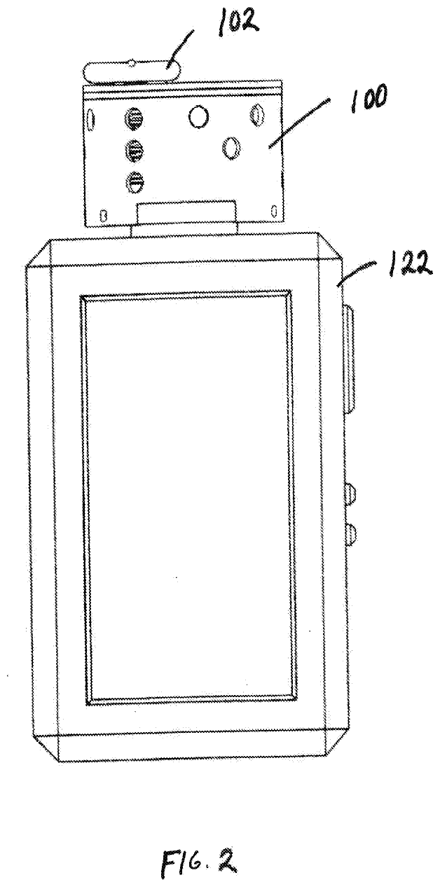

Referring to the Figures, an induction heater module 100 and an adjustment device 102 for induction heating are provided. The induction heater module 100 generally comprises an assembly of an induction heating circuit 104 in electrical communication with a 510 electrical coupling or connector 106 and in electrical communication with an induction coil 108. The induction heater module 100 may receive an incremental ingression insert adjustment device 102 (see generally FIGS. 1-5).

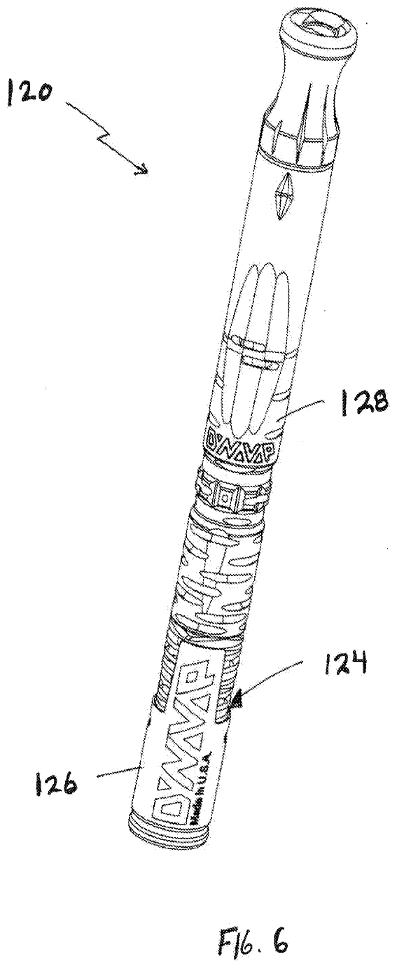

The induction heater module 100 described herein is suitable for use with a thermal extraction device or administration device 120, such as shown in FIG. 6. Suitable examples of such a device 120 are shown and described in U.S. Pat. No. 10,206,425, the entire content of which is hereby incorporated by reference. Generally, a thermal extraction device or administration device 120 includes an extraction chamber 124 which receives material to be extracted, optionally a cap 126 covering the extraction chamber 124, and a stem 128 connected to the extraction chamber for extracting the material.

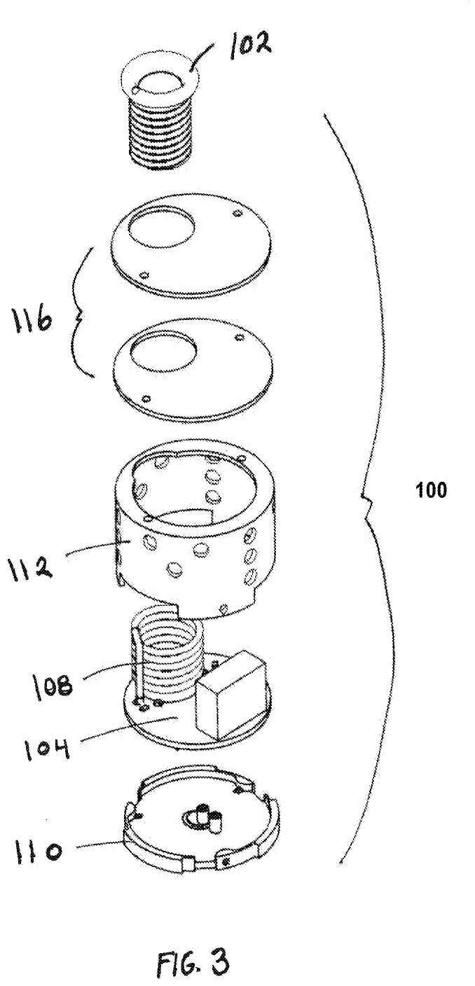

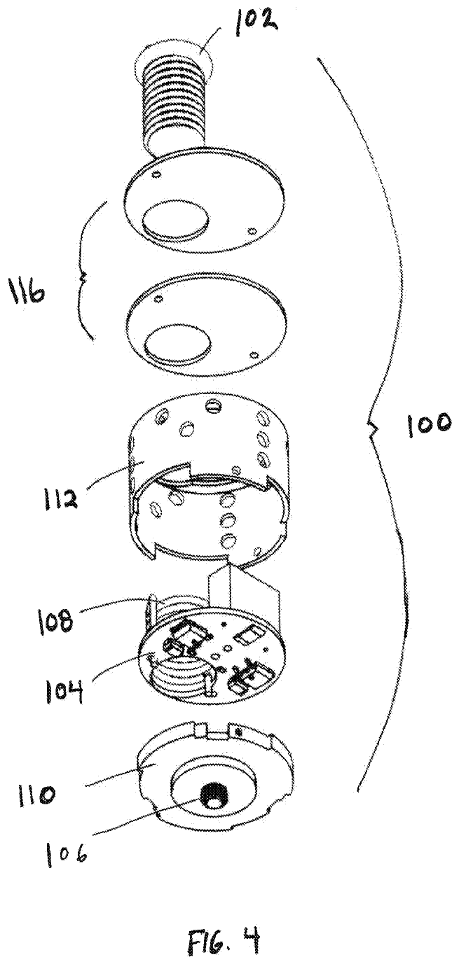

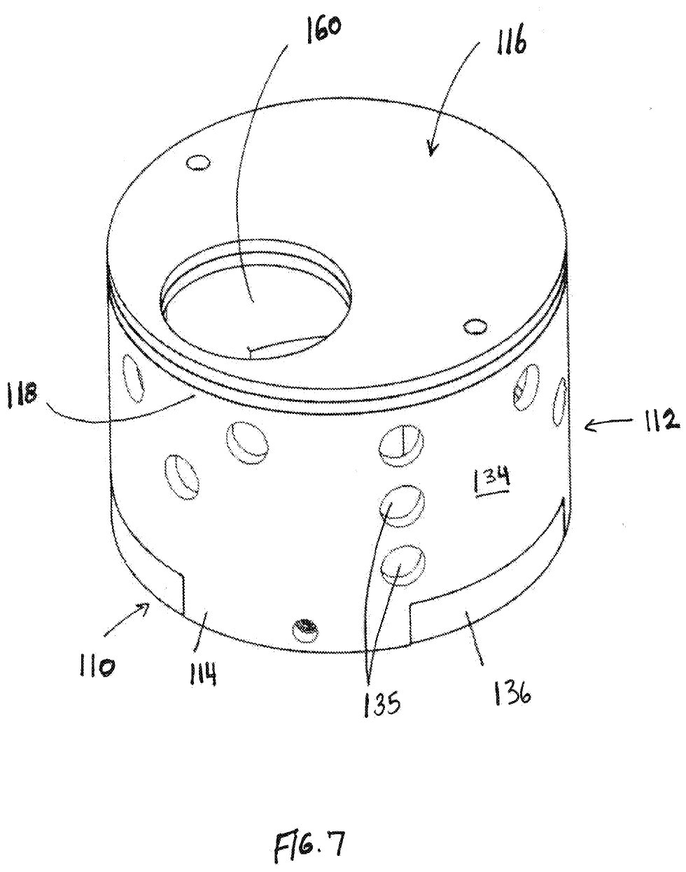

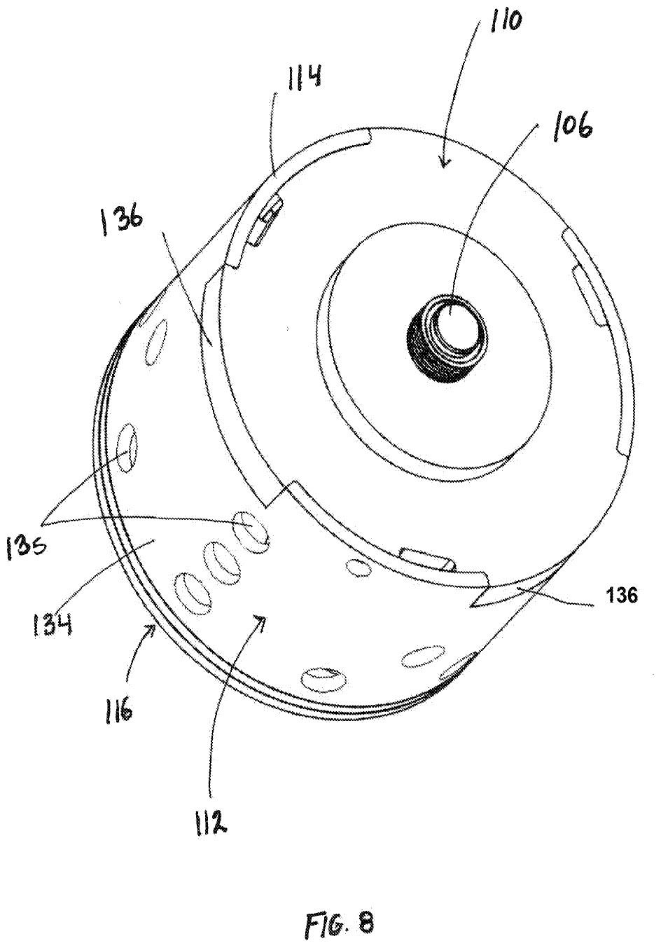

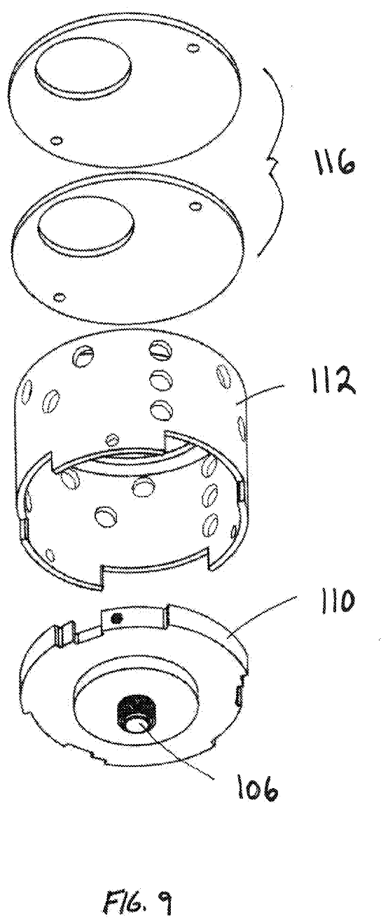

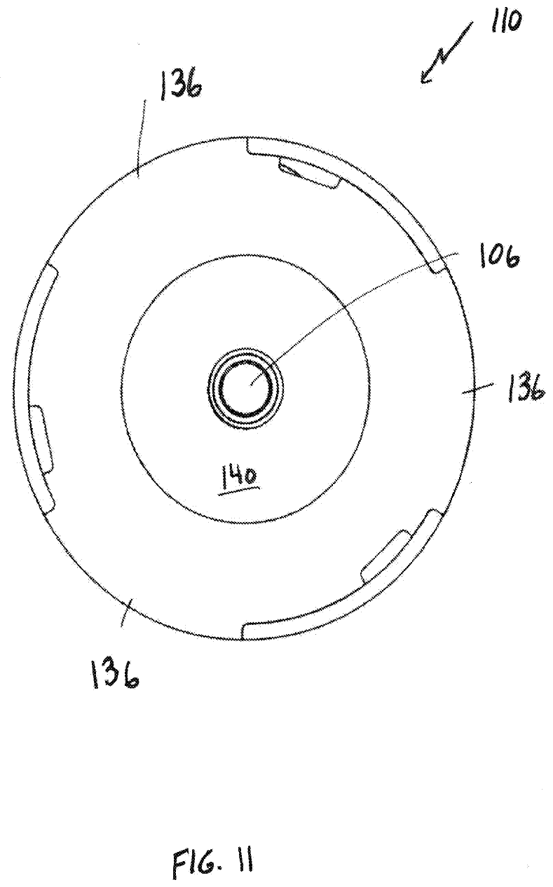

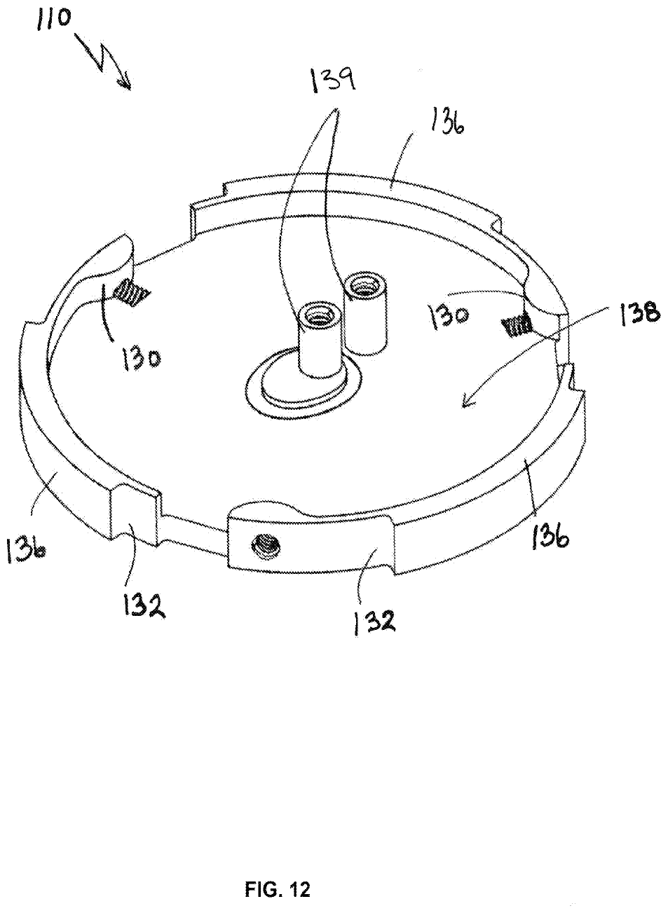

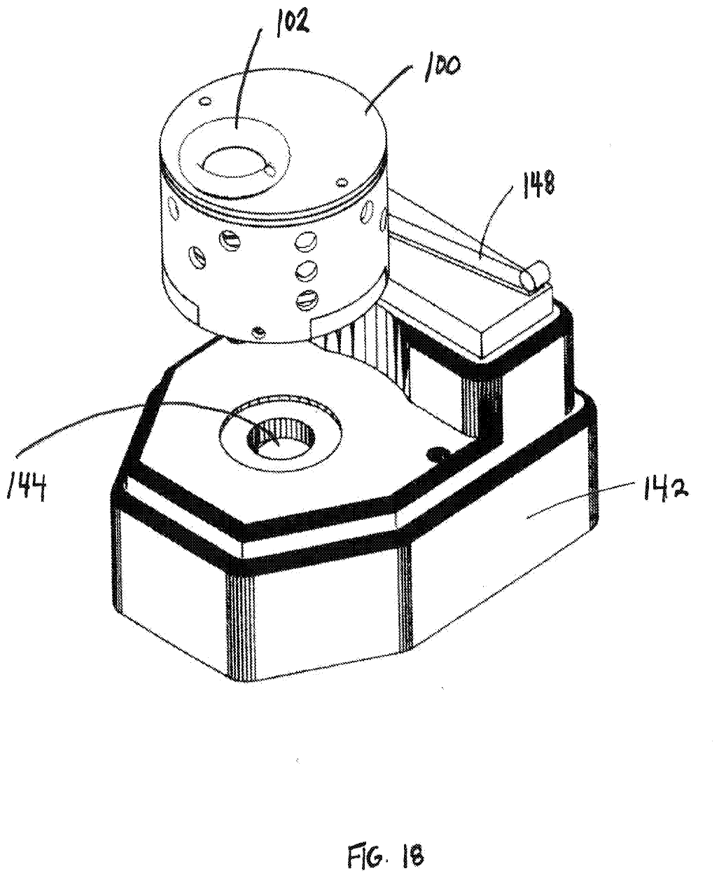

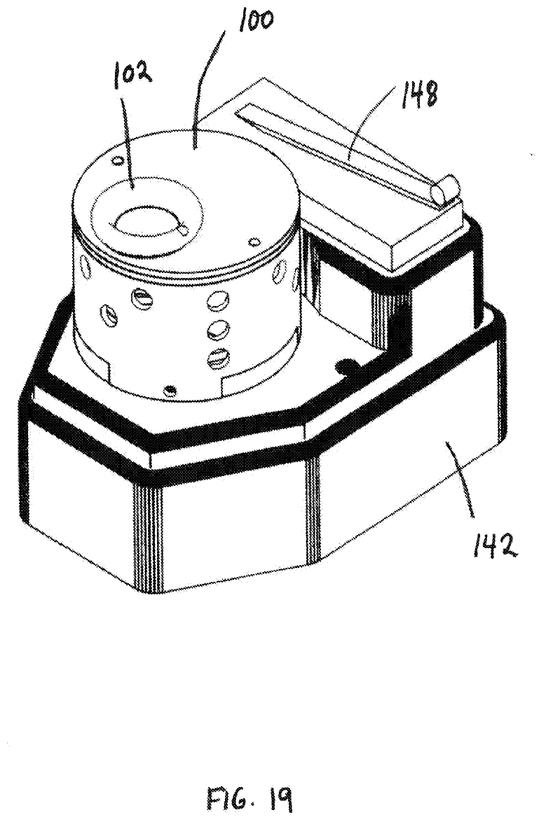

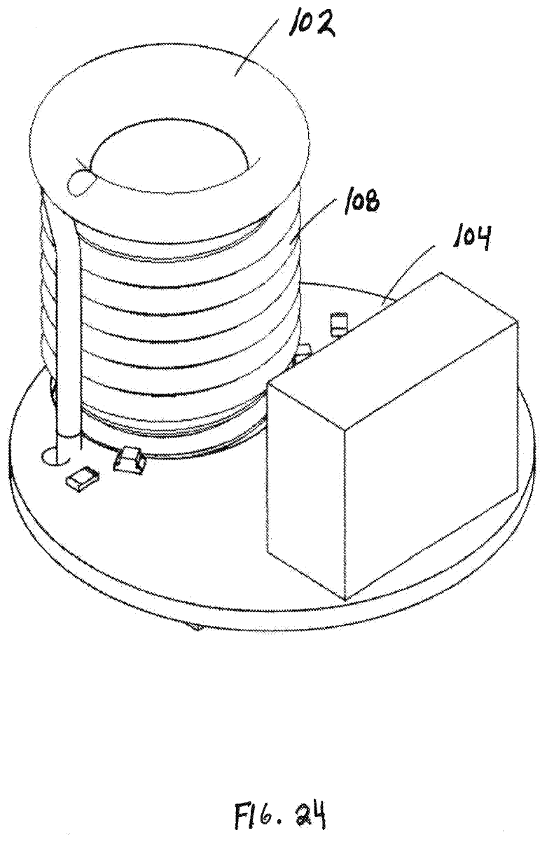

Referring to FIGS. 7-16, the induction heater module 100 is made up of, at least in part, a housing composed of a housing body 112 and a cover 116 on a base 110. More specifically, the induction heater module housing comprises a base 110 couplable to a housing body 112 on a first end or lower end 114, and a cover 116 or lid couplable to the housing body 112 on a second end or upper end 118. Referring to FIGS. 3-4, the housing receives and contains the induction heating circuit 114 and coil 108 therein, as well as at least a portion of the adjustment device or receptacle 102 for receipt of an administration device 120 (see FIG. 24). The base 110 is couplable, via a 510 electrical coupling 106, to a power source 122 (see FIGS. 1-2 and 17-19).

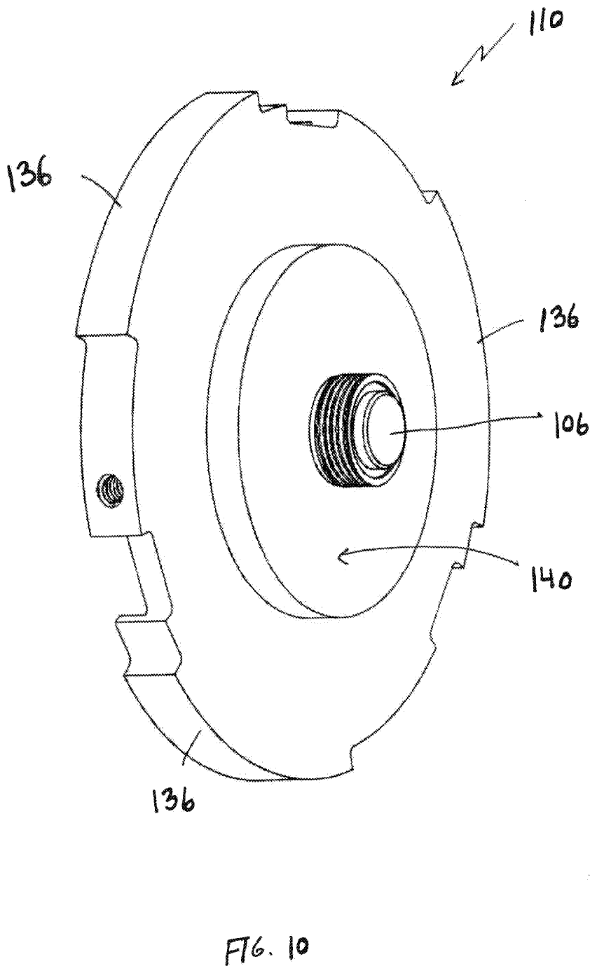

In more detail, the base 110 is composed of a durable material and includes a conductive material or component which is conductive. Referring to FIGS. 10-12, the geometry of the base 110 may include various ramps 130 and other undercut 132 geometry. In the illustrated examples, the base 110 has three lobes 136. However, any number of lobes 136, e.g., three lobes, five lobes, four lobes, two lobes, and so forth, may be acceptable for the purposes provided. The base 110 to which the housing body 112 connects is generally a machined metal (or other suitable material) part; although variations thereon are also acceptable such as but not limited to a molded configuration. The base has a 510 threaded connector 106 which is conductive and allows for connection to/is couplable to the power source 122. An area may also be provided on the upper surface 138 of the base 110 to receive, electrically couple, and/or carry an induction circuit 104. This area may also be connected to the 510 threaded connector 106. In the illustrated embodiment, one or more power input connectors 139 for positive (+) and negative (−) are provided to electrically couple the 510 connector 106 to the induction heating circuit 114 and provide the ability to power the induction heater module 100. The power input connectors 139 may be threaded to allow for a secure connection between the induction heating circuit 114 and the base 110, held in place with screws or other fasteners; although alternative means of connecting and electrically coupling the two components are known and may be substituted in place of the illustrated means. The power input connectors 139 in the illustrated embodiment serve two purposes: a secure mounting connection and electrical connection.

The base 110 geometry enables simplistic manufacturing of a couplable housing body 112. For example, in the illustrated embodiment the base 110 includes three lobes 136. The base 110 geometry, e.g., lobes 136, provide areas which couple to mating housing body 112 geometry (discussed in further detail hereinbelow) at its base or lower end 114 via a press fit configuration, and in one or more examples of embodiments, may have a snap lock interface. Additionally, or alternatively, fasteners 154, or other fastening mechanisms may be used such as tongue and groove, pin, threaded devices, and the like. The cover 116 may be constructed and attached to the housing body 112 in a similar manner.

Regardless of the number of lobes 136, the base 110 configuration involves geometry where the complexity of the manufacturing primarily lives in the metallic conductive base 110 that connects to the power source 122. In other words, the shape of the metallic base 110 is configured to mate with a housing body 112 in a positive locking fashion with a machinable geometry that can be more or less easily created and manufactured utilizing simple tools; for example, a two-axis machine, a lathe with a live tool, a CO₂ laser, a tool with a rotary axis, or the like. In this regard, the induction heater module housing 112 and cover 116 described herein is a two-axis manufacturing-enabled design because the geometry of the base 110 allows the housing body 112, once cut on a two-axis machine, to snap together and/or fasten together with the base 110 for easy assembly. In this manner, the manufacture of housing components is flexible, allowing users to create their own housing body 112 and cover 116 without the need for complex manufacturing equipment.

In one example, the base 110 geometry may be manufactured by use of, for example, a three-axis milling machine; although any form of manufacture which accomplishes the intended purposes would be acceptable.

As indicated, the area provided on the upper surface 138 of the base 110 may receive, electrically couple, and/or carry an induction circuit 104 which may include a circuit board or multiple circuit boards, which in some examples of embodiments may be electrically coupled to an induction coil 108, which may also be carried on the upper surface 138 (see FIGS. 3-4, 24).

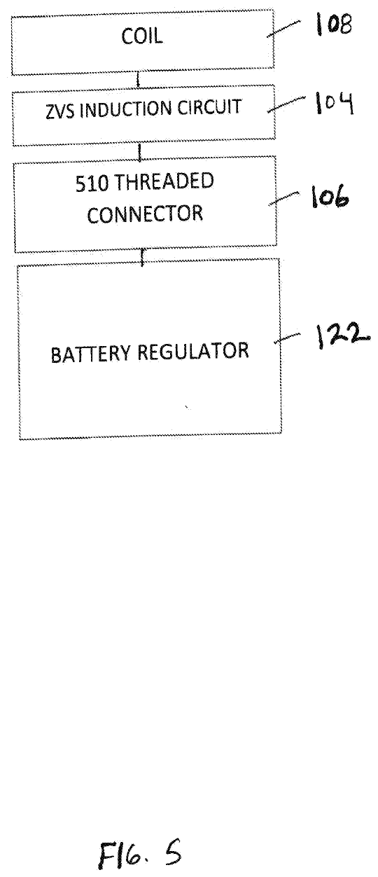

In one example, a ZVS induction heater circuit 104 with an induction work coil 108 is mounted and electrically coupled to the 510 threaded connector 106 (see FIG. 5, 24). The coil 108 may be a single-start thread design. In one or more alternative examples of embodiments, the coil 108 of the induction heater module 100 may be a center wound coil 108, that is a coil 108 with ends terminating on the same side, such as a multi-start thread (e.g., a two-start or double start thread).

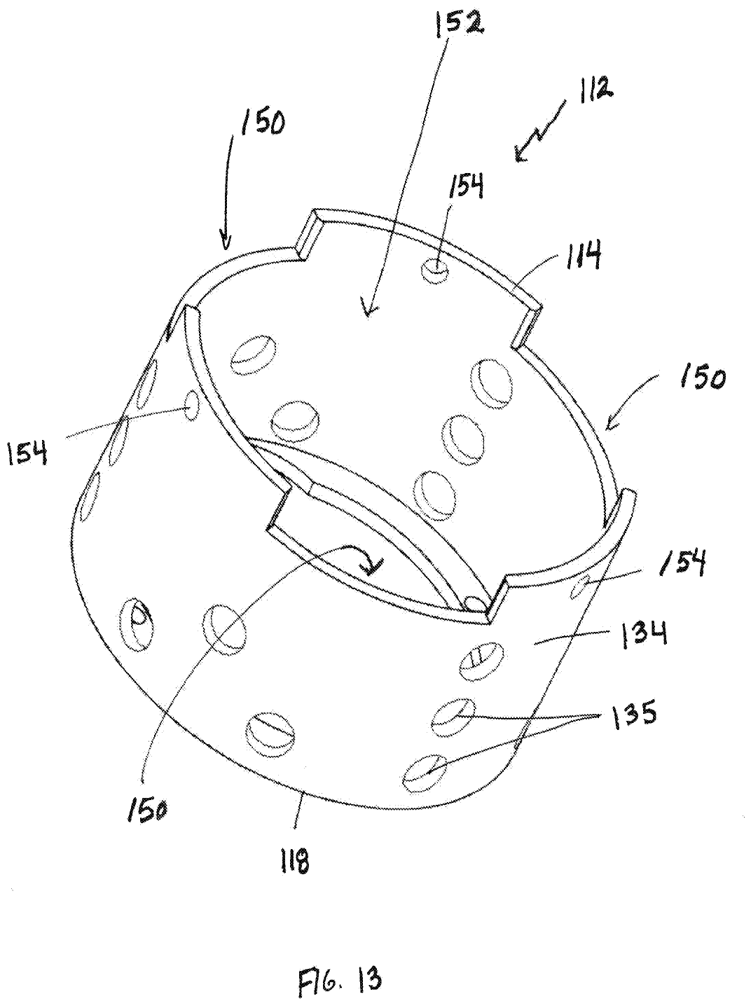

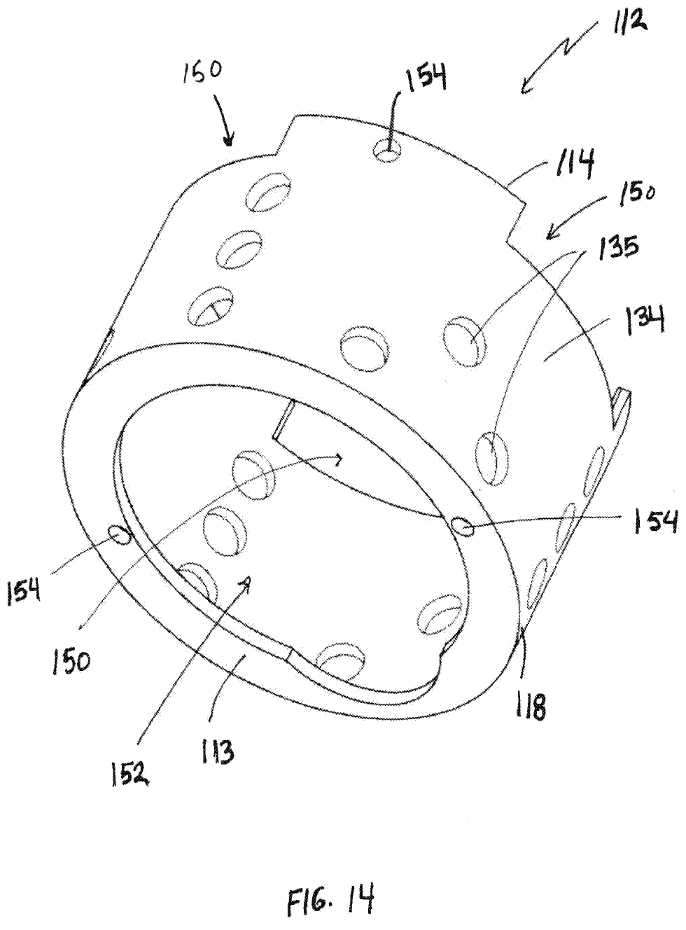

As indicated, the base 110 which carries the 510 threaded connector 106 is coupled to a housing body 112. Referring to FIGS. 13-14, the housing body 112 is generally constructed of one or more sidewalls 134 having mating geometry to the base 110 and cover 116 on opposing ends 114, 118 of the sidewall(s) 134. In the illustrated example, the sidewall 134 is an annular sidewall having one or more recesses 150 on one end 114 of the sidewall 134 which mate with the one or more lobes 136 of the base 110; such that the base 110 covers one end 114 or opening of the housing body 112. The sidewall(s) 134 forms a hollow opening or interior 152 therein or therebetween, which is sized to receive the induction circuit 104, induction coil 108, and adjustment device 102. While in the illustrated example the sidewall 134 a generally hollow cylinder is shown, variations thereon are acceptable as any geometric shape creating an area therein may be suitable for the purposes provided. One or more apertures 135 may be provided in the sidewall(s) 134 which may be used for cooling of the device or other purposes. Fasteners 154 may also be coupled with the sidewall(s) and secure the sidewall(s) to the base. One or more contacts (not shown) may also be provided on the housing body 112 or the base 110.

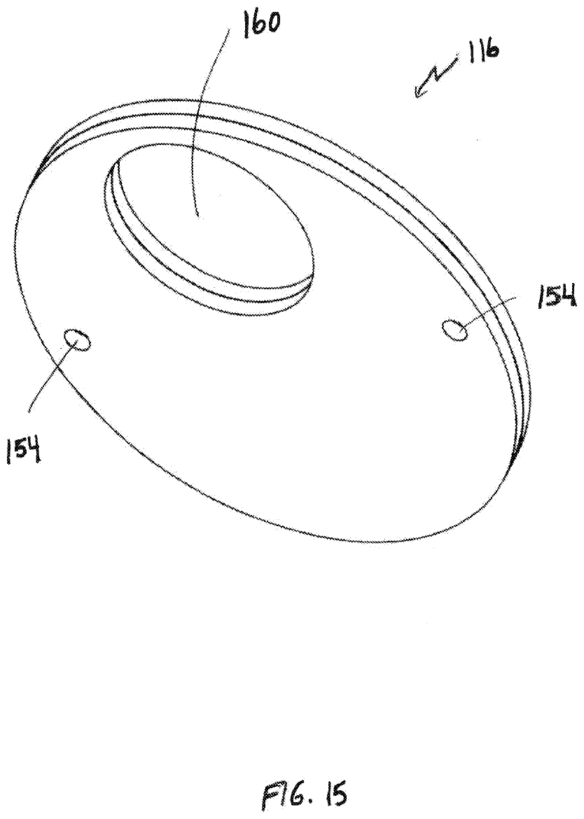

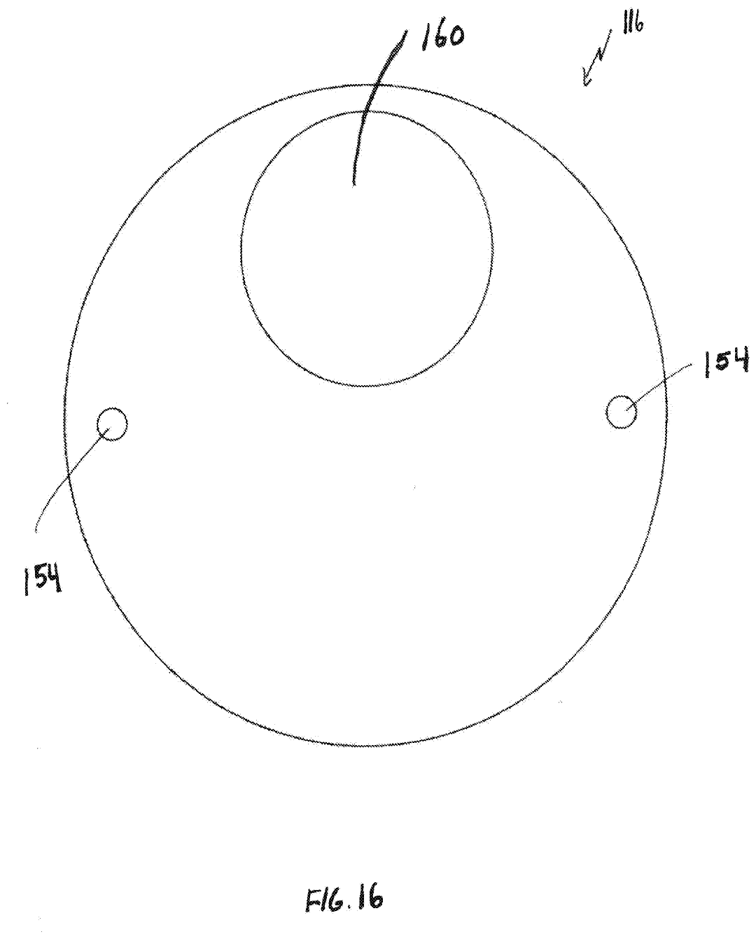

The cover 116 in the illustrated example covers one end 118 of the housing body 112. In this regard, the cover 116 is provided with a mating geometry to the housing body 112 shape and sidewalls 134. In the illustrated embodiment, the cover 116 is attached by one or more fasteners. One or more covers 116 may also be stacked on top of one another and attached by the one or more fasteners to the housing body 112. In this regard, the housing body 112 may have a flange 113 or the like to which the fasteners may be secured or which otherwise may support the cover 116. In alternative examples of embodiments, the cover 116 may have one or more lobes which mate with recesses in the housing body 112. Referring to FIGS. 15-16, an opening or aperture 160 may be provided through the cover 116 to access the hollow interior 152 of the housing, and/or to insert a receptacle for an administration device 120. For example, the aperture 160 may provide access to and/or be aligned with the induction coil 108. The cover 116 may be further attached or alternatively attached by other fastening means now known or future developed.

As shown in the Figures, the 510 threaded connector 106 is positioned on a lower surface 140 of the base 110 and may be integral therewith or a separate component attached thereto. The 510 threaded connector 106 allows the induction heater module 100 to be connected to and powered by any 510 threaded device. The 510 thread is a universal thread design. The reason that this power coupling has been given the name 510 is owing to its length which is 5 mm and the 10 screw threads that form part of it. Attachments compatible with 510 can be switched with one another. Therefore, an induction heater module 100 with a 510 threaded connector 106 can be used and interchanged without the need to purchase a new battery or power source 122.

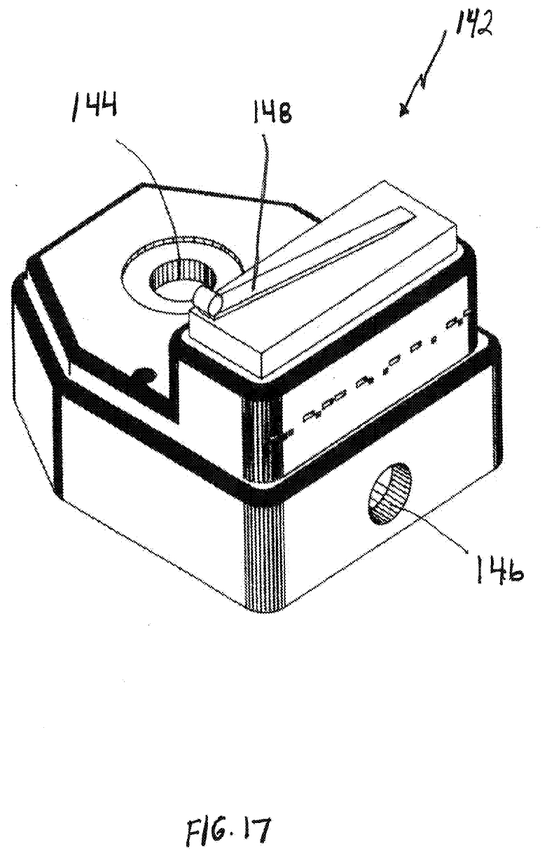

Examples of 510 power source devices 122 may include, but are not limited to, box mods, 510 battery regulators or other power supplies and controllers such as the base station 142 shown in FIGS. 17-19. Generally, the power source 122 comprises a mating 510 threaded connector 144 such as a receptor, a source of power such as an AC or DC plug or battery (an electrical coupling 146, such as a cord receptor for a plug is shown in FIG. 17), circuitry (not shown) coupling the source of power 146 to the 510 threaded connector 144, and a switch 148 for turning the power source 122 on and off. Some 510 power supplies 122 are provided with the feature of variable temperature settings while others come with preset temperatures.

In use, an administration device 120 can be inserted into the induction work coil 108 within the induction heater module 100, and if present inserted into the adjustment device 102 which is within the induction work coil 108 (discussed below). The attached power source 122 will then activate the ZVS induction circuit 104 and begin heating the administration device 120 via induction. In one or more examples of embodiments, the induction heater module 100 may operate in a range of between 2-15V and have a heat time of less than 10 seconds, although variations thereon are also acceptable.

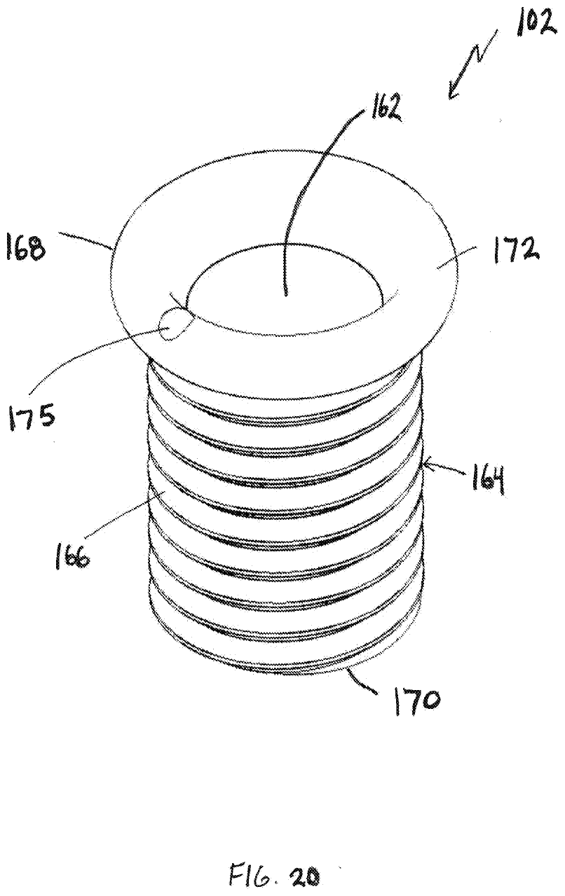

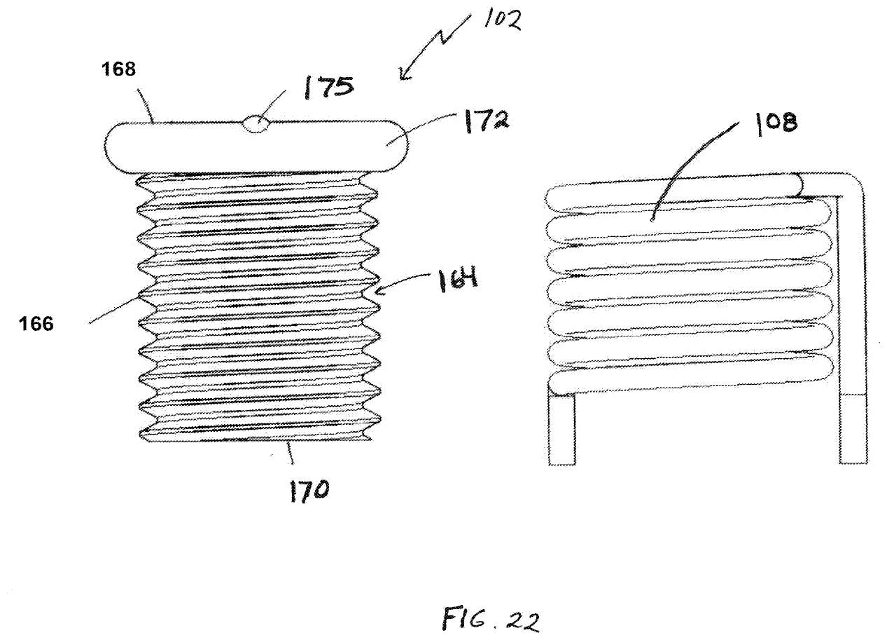

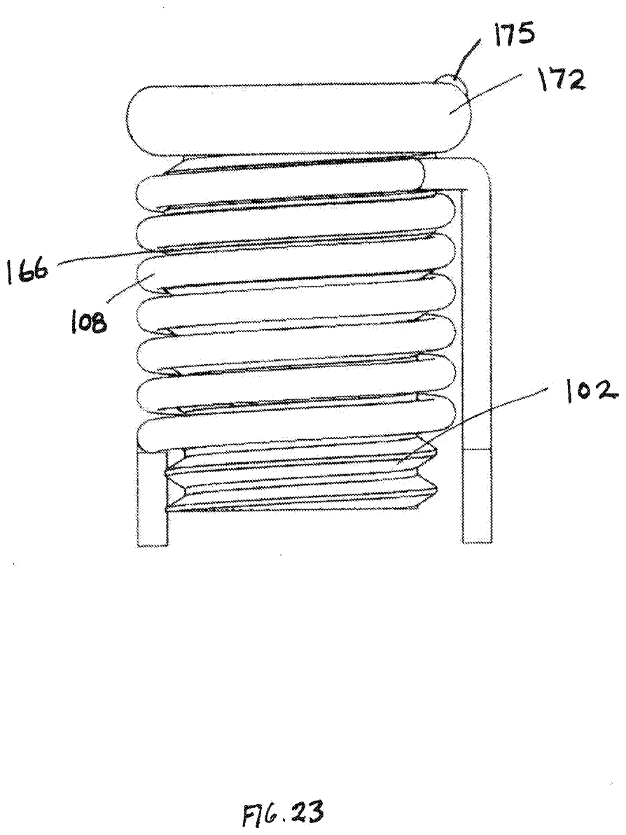

In one more or more examples of embodiments, an adjustment device or induction heater depth control device 102 is further disclosed. More specifically, an incremental ingression insert adjustment device 102 for an induction heater is disclosed. Referring to FIGS. 20-24, the device comprises a receptacle 102 having an inner recess 162 for receipt of an administration device 120 to be heated, and an exterior surface 164 comprising a thread 166 having a pitch aligned with the pitch of an induction coil 108 of an induction heater. The device or receptacle 102 is rotatably or insertably engaged with the induction coil 108.

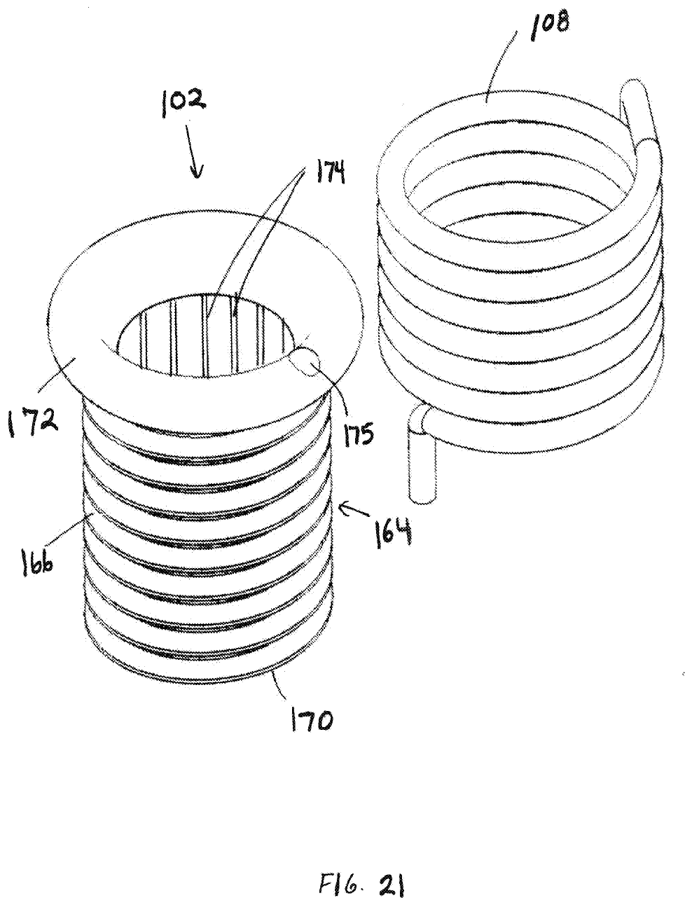

In one or more examples of embodiments, the receptacle 102 may be a cup comprising a generally smooth inside wall surrounding the inner recess 162 (see FIG. 20), an outside wall comprising a surface 164 consisting of threads 166, and optionally a lip or curved lip or stop 172 on top. Alternatively, the interior recess 162 of the receptacle 102 may have one or more spaced apart ribs 174 for spacing an administration device 120 inserted therein away from the inner sidewalls or surface of the receptacle (see FIG. 21). As indicated, the inner recess 162 is of sufficient size to accommodate an administration device 120, such as a thermal extraction device, to be at least partially inserted into the recess of the receptacle or adjustment device. The receptacle 102 has a top 168 with an opening accessible to the recess 162 for receiving an administration device 120 and a bottom 170 which may be closed forming a resting location for the administration device 120 inserted into the receptacle 102. The receptacle 102 has a height or depth suitable for receiving at least a portion of the administration device 120, and preferably the portion to be heated by the induction heater or induction heater module 100. The width and/or diameter of the receptacle inner recess 162 is sufficient to receive and partially retain an administration device 120 therein. The threaded outside wall of the receptacle 102 mates with the wire induction coil 108, allowing the receptacle 102 to thread into the coil 108 just as a screw would thread into a nut. The coil 108 supports the receptacle 102 and holds it firm, while still allowing the receptacle 102 to be adjusted up or down by turning the receptacle, or alternatively pressing or pulling the receptacle, further into or out of the coil 108. The top 168 of the receptacle 102 may have a rounded lip, a flange, or stop 172 for limiting the insertion depth of the receptacle 102 into the induction coil 108, and for ease of adjustment with fingers. To this end, the lip, flange, or stop 172 may be fluted, knurled, or include other geometry for each of grip and rotation or insertion into the induction coil 108.

While specific examples are described, alternative geometries accomplishing the same or similar purposes would also be acceptable.

As indicated the exterior surface 164 of the receptacle 102 or adjustment device has a thread 166. The thread 166 may be helical. The thread 166 has a depth or height generally corresponding to the height of the receptacle 102 although variations thereon may be acceptable. The thread 166 is configured to engage the induction coil 108. More specifically, the thread 166 rotatably engages the induction coil 108. Alternatively, the thread 166 may be pressed into the coil 108. In this manner, the induction coil 108 acts like a mating thread. The thread 166 on the exterior surface 164 of the receptacle 102 provides a mechanism to specifically space the coil 108. In this regard, the thread 166 on the exterior surface 164 may generally match the pitch of the induction coil 108. Further, the elasticity of the coil 108 retains/holds the receptacle 102 in place by this interaction or engagement with the thread 166. Moreover, the thread 166 which mates with the coil 108 provides a specific means to control and adjust the depth of the receptacle 102 in the induction coil 108 and consequently the position of the administration device 120 inserted therein relative to the induction coil 108, thereby providing an adjustment device for incremental adjustment of ingress or insertion depth.

The adjustment device or receptacle 102 may be formed of any number or variety of materials. In one or more examples of embodiments, the receptacle 102 may be formed of materials which are heat resistive (and in some instances with a very high melting point). In other examples of embodiments, the materials are easily cleaned. The material also includes a degree of durability. In another example of embodiments, the device or receptacle 102 may be formed of a material(s) which is non-conductive; although it is contemplated that conductive material may be suitable in certain embodiments. In the example provided, the device or receptacle 102 is a threaded cup made of quartz, borosilicate, or similar material suitable for high temperature use. However, as indicated any material suitable for the purposes provided may be acceptable.

As indicated, in one or more examples of embodiments, the induction coil 108 may be a center wound coil, that is a coil 108 with ends terminating on the same side, such as a multi-start thread (e.g., a two-start or double start thread). The pitch of the thread 166 on the receptacle 102 or cup may then be constructed to correspond with this coil 108, or in the case of a single-start thread design, the pitch of the single-wound thread 166.

In addition, the receptacle or cup 102 may have one or more spaces, detents, extensions, visual indicators or other devices 175 configured to provide a tactile and/or audible signal as the cup is rotated or inserted, communicating a location of the insertion or withdrawal or position of the receptacle 102.

When the thermal extraction device or administration device 120 is inserted into the cup 102 and the cup 102 is inserted into the coil 108, the administration device 120 can be raised or lowered by threading the cup 102 into or further out of the coil 108. Thus, in use, rotation or insertion of the receptacle 102 relative to the coil 108 causes the movement of the receptacle 102 relative to the coil 108 thereby inserting the receptacle 102 further into the coil 108 (until it reaches the stop 172 on the top 168 of the receptacle 102 caused by, for example, the upper lip, and consequently a majority or the entirety of the receptacle 102 and inserted administration device 120 portion are within the coil 108); or withdrawing the receptacle 102 from the coil 108 (i.e., moving the bottom 170 of receptacle 102 within the coil 108 such that a smaller portion of the receptacle 102, and consequently the administration device 120, is within the coil 108). This rotation or insertion/withdrawal allows for an extensive amount of adjustment and tunability, as the user can precisely control the depth at which the receptacle 102 is provided within the coil 108 (and consequently the depth of the administration device 120 within the coil 108).

Changing the height of the administration device 120 relative to the height of the heating coil 108 moves the focal point of the applied heat on the administration device 120. In doing so, a user can find the ideal height of the administration device 120 to their own heating preference. The user can also change the height as needed to further optimize the heating experience.

Accordingly, the adjustment device or receptacle 102 disclosed herein provides a means to control the insertion depth of the administration device 120 or provide an incremental ingression insert adjustment into and out of the induction coil 108; thereby allowing careful and tunable control of the heating of the administration device 120.

As utilized herein, the terms “approximately,” “about,” “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the invention as recited in the appended claims.

It should be noted that references to relative positions (e.g., “top” and “bottom”) in this description are merely used to identify various elements as are oriented in the Figures. It should be recognized that the orientation of particular components may vary greatly depending on the application in which they are used.

For the purpose of this disclosure, the term “coupled” means the joining of two members directly or indirectly to one another. Such joining may be stationary in nature or moveable in nature. Such joining may be achieved with the two members or the two members and any additional intermediate members being integrally formed as a single unitary body with one another or with the two members or the two members and any additional intermediate members being attached to one another. Such joining may be permanent in nature or may be removable or releasable in nature.

It is also important to note that the construction and arrangement of the system, methods, and devices as shown in the various examples of embodiments is illustrative only. Although only a few embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts or elements show as multiple parts may be integrally formed, the operation of the interfaces may be reversed or otherwise varied, the length or width of the structures and/or members or connector or other elements of the system may be varied, the nature or number of adjustment positions provided between the elements may be varied (e.g. by variations in the number of engagement slots or size of the engagement slots or type of engagement). The order or sequence of any process or method steps may be varied or re-sequenced according to alternative embodiments. Other substitutions, modifications, changes and omissions may be made in the design, operating conditions and arrangement of the various examples of embodiments without departing from the spirit or scope of the present inventions.

While this invention has been described in conjunction with the examples of embodiments outlined above, various alternatives, modifications, variations, improvements and/or substantial equivalents, whether known or that are or may be presently foreseen, may become apparent to those having at least ordinary skill in the art. Accordingly, the examples of embodiments of the invention, as set forth above, are intended to be illustrative, not limiting. Various changes may be made without departing from the spirit or scope of the invention. Therefore, the invention is intended to embrace all known or earlier developed alternatives, modifications, variations, improvements and/or substantial equivalents.

The technical effects and technical problems in the specification are exemplary and are not limiting. It should be noted that the embodiments described in the specification may have other technical effects and can solve other technical problems.

1. An induction heater module comprising:
a 510 threaded connector couplable to a power source;
an induction circuit electrically connected to the 510 threaded connector; and
an induction coil electrically connected to the induction circuit.

2. The induction heater module of claim 1, further comprising a housing with a base having the 510 threaded connector thereon.

3. The induction heater module of claim 2, wherein the base is configured for coupling to a housing body which is couplable to a cover.

4. The induction heater module of claim 1, wherein an incremental ingression insert adjustment device is provided at least partially into the induction coil.

5. A base of an induction heater module comprising:
a 510 threaded connector couplable to a power source and electrically couplable to an induction heating circuit and induction coil;
wherein the base comprises a geometry which enables press tit attachment of a housing body.

6. An incremental ingression insert adjustment device comprising:
a receptacle having an inner recess for receipt of an administration device; and
an exterior surface comprising a thread having a pitch aligned with the pitch of an induction coil of an induction heater;
wherein the receptacle is rotatably or insertably engaged with the induction coil.

7. The adjustment device of claim 6, wherein the receptacle has a stop on a portion of the device for limiting insertion of the receptacle into the induction coil.

8. The adjustment device of claim 6, wherein the receptacle has a tactile or visual indicator of relative position.

9. The adjustment device of claim 6, wherein the receptacle has one or more ribs on an inner surface of the inner recess.

10. An induction heater module housing comprising:
a base with an electrical coupling, the base also couplable to a housing body on a first end of the housing body, and a cover couplable to the housing body on a second end of the housing body.

11. The induction heater module housing of claim 10, wherein the housing receives an induction heating circuit and coil, and at least a portion of a receptacle configured for receipt of an administration device.

12. The induction heater module housing of claim 10, wherein the housing further comprises an incremental ingression insert adjustment device at least partially received within the housing body.
